SEQUENCE LISTING (17 sequences)

SEQ ID NO: 1 - DNA, 612 bp, Homo sapiens
  CDS (1)..(612)
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 2 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - DNA, 522 bp, Homo sapiens
  CDS (1)..(522)
acccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 4 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 5 - DNA, 621 bp, Homo sapiens
  CDS (1)..(621)
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 6 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 7 - DNA, 531 bp, Homo sapiens
  CDS (1)..(531)
acccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 8 - protein, 177 aa, Homo sapiens
TPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 9 - DNA, 525 bp, Homo sapiens
  CDS (1)..(525)
atgacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 10 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 11 - protein, 208 aa, Mus musculus
MAQLSAQRRMKLMALQLLLWQSALWSGREAVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLA

SEQ ID NO: 12 - protein, 214 aa, Rattus norvegicus
MKLMALQLLLWHSALWSGQEAIPLLTVSSLPPSLPLPRSFLLKSLEQVRKIQARNTELLEQLCATYKLCHPEELVLFGHSLGIPKASLSSCSSQALQQTKCLSQLHSGLFLYQGLLQALAGISSELAPTLDMLHLDVDNFATTIWQQMESLGVAPTVQPTQSTMPIFTSAFQRRAGGVLVTSYLQSFLETAHHALHHLPRPAQKHFPESLFISI

SEQ ID NO: 13 - protein, 194 aa, Felis catus
KLMALQLLLWHSALWMVQEATPLGPTSSLPQSFLLKCLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPTQGTMPTFTSAFQRRAGGTLVASNLQSFLEVAYRALRHFTKP

SEQ ID NO: 14 - protein, 195 aa, Bos taurus
MKLMVLQLLLWHSALWTVHEATPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 15 - protein, 195 aa, Sus scrofa
MKLMALQLLLWHIALWMVPEAAPLSPASSLPQSFLLKCLEQVRKIQADGAELQERLCATHKLCHPQELVLLGHSLGLPQASLSSCSSQALQLTGCLNQLHGGLVLYQGLLQALAGISPELAPALDILQLDVTDLATNIWLQMEDLRMAPASLPTQGTVPTFTSAFQRRAGGVLVVSQLQSFLELAYRVLRYLAEP

SEQ ID NO: 16 - protein, 7 aa, Artificial Sequence
  Description of Artificial Sequence Synthetic peptide for GCSF
LGHSLGI

SEQ ID NO: 17 - protein, 174 aa, Artificial Sequence
  Description of Artificial Sequence Synthetic polypeptide variant of human G-CSF
APTTRASSLPQSFLLKSLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2012/0070403 A1
(43) Pub. Date: Mar. 22, 2012

(54) USE OF G-CSF FOR THE EXTENSION OF THE THERAPEUTIC TIME-WINDOW OF THROMBOLYTIC STROKE THERAPY

(75) Inventors: Marc Fisher, Newton, MA (US); Armin Schneider, Heidelberg (DE)

(73) Assignee: SYGNIS BIOSCIENCE GMBH & CO. KG, Heidelberg (DE)

(21) Appl. No.: 13/201,866

(22) PCT Filed: Feb. 17, 2010
(86) PCT No.: PCT/US10/24426

§ 371 Date: Nov. 30, 2011

Related U.S. Application Data
(60) Provisional application No. 61/153,079, filed on Feb. 17, 2009.

(51) Int. Cl. A61K 38/19; A61P 25/00; C12N 9/48; A61P 9/00; C12N 9/72; C12N 9/70

(52) U.S. Cl. 424/85.1; 435/215; 435/216; 435/212

(57) ABSTRACT

The present invention relates to the use of G-CSF and derivatives thereof for extending the therapeutic window of subsequent thrombolytic treatment of acute stroke, and thereby, allowing the diagnostic examinations which are necessary prior to the thrombolytic treatment in order to avoid hemorrhagic and other severe adverse side effects of the thrombolysis.

### CROSS-REFERENCES TO RELATED APPLICATIONS

The present application claims priority to U.S. Ser. No. 61/153,079, filed Feb. 17, 2006, herein incorporated by reference in their entirety.

### FIELD OF THE INVENTION

The present invention relates to the use of Granulocyte Colony Stimulating Factor (G-CSF) polypeptide in the prevention of neuronal cell death in the infarct penumbra after acute stroke. More particularly, the invention provides methods of enhancing the therapeutic window for thrombolytic treatment after acute stroke by the preceding administration of G-CSF polypeptide in conjunction with a subsequent thrombolytic therapy.

### BACKGROUND OF THE INVENTION

Granulocyte colony stimulating factor (G-CSF) was originally identified as a hematopoietic factor in the myeloid lineage responsible for the generation of neutrophilic granulocytes. Recently, the presence and activity of this factor in the central nervous system was identified G-CSF and its receptor are up-regulated after cerebral ischemia, G-CSF acts anti-apoptotically on neurons, passes the intact blood-brain barrier, and reduces infarct size in experimental stroke models (Schneider et al., J Clin Invest. 2005, 115:2083; Zhao et al., Exp Neurol. 2007, 204:569; Schabitz et al., Stroke 2003, 34:745; Six et al., Eur J Pharmacol. 2003, 458:327; Shyu et al., Circulation 2004, 110:1847; Gibson et al., 2005, 25:431; Komine-Kobayashi et al., J Cereb Blood Flow Metab. 2006, 26:402; Schneider et al., BMC Biol. 2006, 4:36). This has led to a number of smaller clinical trials in acute ischemic stroke patients (reviewed in Schabitz et al., Stroke 2006, 37:1654; Schabitz et al., Trends Pharmacol Sci. 2007, 28:157). However, although meta-analysis of published data supports the broad basis for efficacy of this factor in experimental stroke models (Minnerup et al., Stroke 2008, 39:1856), the majority of experiments were done using transient ischemic models. In particular, no published data exist on embolic models.

Thrombolysis with recombinant tissue plasminogen activator (rt-PA) remains the only approved acute stroke therapy until now. Unfortunately, the use of rt-PA is limited by a relatively narrow time window. Efficacy was recently demonstrated up to 4.5 h following onset of stroke symptoms, but efficacy decline rapidly over time (Hacke et al., Lancet 2004, 363:768; Hacke et al., N Engl J Med. 2008, 359:1317). The biological reason for the reduced therapeutic efficiency over time likely lies in the progressing deterioration of cell viability with ongoing ischemia/hypoxia in hypoperfused brain areas. This may be paired with generation of free radicals during reperfusion (i.e., reperfusion injury). Clinically, this concept is supported by data that suggest that the presence of a perfusion/diffusion (PWI/DWI) mismatch on MRI identifies patients where thrombolysis may be efficacious later in the therapeutic time window (Fisher et al. Cerebrovasc Dis. 2006, 21 Suppl 2:64).

A strategy to extend the time window for thrombolysis may be to protect tissue at risk identified as the PWI/DWI mismatch region. Proof-of-concept for this hypothesis has been demonstrated with normobaric hyperoxia treatment (Henninger et al. J Cerb Blood Flow Metab. 2007, 27:1632) and stimulation of the sphenopalatine ganglion (Henninger and Fisher Stroke 2007, 38:2779).

### BRIEF SUMMARY OF THE INVENTION

Cerebral infarcts caused by stroke comprise the infarct core (already irreversibly injured tissue) and the penumbra (tissue at risk but still salvageable). Thrombolysis, particularly with tissue plasminogen activator (t-PA), is known as an effective treatment of acute ischemic stroke but only if therapy is initiated within a short time period (therapeutic window) after the onset of stroke. The volume of salvageable penumbra tissues decreases strongly continuously over timewithin the first hours of cerebral ischemia. Thereafter, the thrombolytic establishment of reperfusion is ineffective in preventing further neuronal cell death and ameliorating the clinical outcome or is even harmful. t-PA has to be administered within the first 4.5 h preferably 3 h, after stroke onset, whereas this time period is sometimes extended up to a total of 6 h by the physicians.

For this reason, early thrombolytic intervention is usually desired. On the other hand however, thrombolytic intervention may have severe hemorrhagic adverse side effects which worsen the clinical outcome of the stroke patient. Therefore, thrombolytic treatment requires neuroimaging to exclude a hemorrhage and assessment of basic coagulation parameters prior to administration of the thrombolytic agent. During that time however, neuronal cell death in the infarct penumbra continues and the therapeutic window for thrombolysis might close.

There is a need for a method or an agent capable to halt the neuronal cell death in the penumbra (“penumbra freezing”) soon after the onset of the stroke and, thereby, extending the therapeutic window for later thrombolytic treatment which allows for the necessary careful diagnostic examinations and treatment decisions.

The inventors found that G-CSF when administered in a stroke model is capable to preserve the penumbra region and, thereby, prevent further extension of the infarct size. It is well accepted in the art that the extent of preserved penumbra tissue is crucial for the beneficial effect of a thrombolytic reperfusion. Since G-CSF is safe in acute ischemic stroke patients, and at least in animal models there is no indication that it might cause intracerebral hemorrhage, or increase the risk of systemic bleeding, it can be administrated to the stroke patient immediately with the begin of the intensive care and without extensive prior diagnostic examinations and even before admission in or transport to the hospital given by paramedicals or other qualified health professionals. G-CSF can be considered as an emergency drug that could be given in the ambulance to prolong the time-window for, and possibly improve outcome after thrombolysis, e.g by t-PA.

The present invention relates to the use of G-CSF for extending the therapeutic window of subsequent thrombolytic treatment of acute stroke, allowing the necessary pre-thrombolysis diagnostic examinations.

One aspect are methods of treating a patient suffering from acute stroke, comprising initial G-CSF administration, followed by diagnostic examinations, whereas said examinations allows the decision if a thrombolytic therapy is suitable to the patient, and, optionally, based on the results of the diagnostic examination, followed by a thrombolytic treatment. Such diagnostic examinations can be e.g. the exclusion of a hemorrhagic stroke, which is a contra-indication for a thrombolytic therapy.

### BRIEF DESCRIPTION OF THE DRAWINGS

FIG. 1: Infarct volume at 24 h after induction of an embolic ischemia by single clot injection. Shown are edema corrected volumes obtained from TTC-stained sections. Rats were treated with G-CSF at 1 h post clot injection (intravenously) and 4 h post clot injection (intraperitoneal), 120 μg/kg body weight each. G-CSF treatment resulted in significantly smaller infarcts compared to the vehicle group (p<0.05).

FIG. 2: Spatiotemporal evolution of diffusion-weighted lesion within sMCAO model. Rats were subjected to permanent filament occlusion of the MCA, and monitored for 3 h after occlusion for the evolution of the diffusion-weighted lesion. G-CSF orvehicle solution were given at 60 min and at 4 h after occlusion onset. The 60 min dose was started before image acquisition at the 60 min time point. There were no statistical between- or within-group differences in CBF deficit. CBF was significantly larger than ADC at all time points except for 120 and 180 min in the vehicle group. The G-CSF group showed significantly smaller ADC volumes than the vehicle group starting at 90 min. Final infarct volume was also significantly smaller in the G-CSF group compared to the vehicle group (p=0.007). Shown are means+/−SEM; *: p<0.05; PWI volume (A), DWI and final infarct (B), absolute (C) and relative mismatch (D).

FIG. 3: Alignment of G-CSF peptide sequences of various species (human (SEQ ID NO: 6), mouse (SEQ ID NO: 11), rat (SEQ ID NO: 12), feline (SEQ ID NO: 13), bovine (SEQ ID NO: 14), and pig (SEQ ID NO: 15)) shows the position of strongly and less conserved amino acids. Evolutionary strongly conserved amino acids are generally thought to be of major importance for the structure and function of the protein.

### DETAILED DESCRIPTION OF THE INVENTION

The inventors describe a finding that makes G-CSF ideally suited as a time-window extender in stroke treatment for any further therapy, preferably, thrombolytic stroke therapy (e.g. with rt-PA).

This is a very useful application, as a major issue that limits the usefulness of thrombolytic stroke therapy, e.g. rt-PA therapy is the limited time window due to loss of efficacy with time. rt-PA has to be administered usually during the initial 3 to 4.5 h after onset of the stroke based on clinical studies. Occasionally, it might be given by some physicians within up to 6 h. Since the possibility of hemorrhagic side effects of rt-PA has to be excluded for the individual patient to avoid worsening of the situation, it is often difficult to enable a safe thrombolytic therapy during this time-window. G-CSF could be given very soon after the suspicion of a cerebral insult has occurred, as it does not complicate a possible hemorrhagic stroke and as it is well-tolerated even in high doses.

The finding relates to the fact that in an animal model of stroke, permanent filament occlusion, G-CSF keeps the diffusion-weighted deficit stable in the presence of an ongoing ischemia. Such an effect is also known as “penumbra freezing”. This means that damage to brain tissue can be delayed until a thrombolytic therapy can be applied to reopen the occluded vessels. In the cases where G-CSF alone might be not sufficient to enable complete recovery from the stroke, the unexpected finding according to the invention enables a combinational or consecutive therapy comprising an initial step of G-CSF administration to the subject and a later step of administration of an thrombolytic agent, e.g. rt-PA. The earlier G-CSF administration allows for a postponed onset of thrombolytic therapy within the first several days, preferably within the first 24 h, more preferably within the first 12 h after onset of the stroke. This allows for a closer diagnostic examination of the patient after stroke or after suspicion of stroke to ensure a safe and effective additional thrombolytic therapy. The advantage of such a combination of an early G-CSF administration and a postponed thrombolytic therapy (e.g. rt-PA administration) is to the inventors knowledge not disclosed previously.

Unexpectedly, G-CSF was effective in preserving the penumbra tissue even during the time the vessel was occluded.

According to the invention, G-CSF administration is started during the first 12 h, preferably during the first 6 h, and more preferably during the first 3 h after onset of the stroke. Preferred uses of G-CSF could be up to a time window of 24 h in doses of at least 10 μg/kg body weight, at least 90 μg/kg body weight, or at least 130 μg/kg body weight given intravenously (i.v.) or subcutaneously (s.c.) over 1-24 h.

According to the invention, it is included that the administration of G-CSF may either be completed before the administration of the thrombolytic agent or may be continued after the administration of the thrombolytic agent. Furthermore, it is also included within the present invention that G-CSF may be administered only once. Alternatively, G-CSF may also be administered in at least two separate steps.

Preferably human recombinant G-CSF, such as Filgrastim, is used according to the invention. Also functional G-CSF derivatives which are know to the person skilled in the art can be used according to the invention.

The method according to the invention is suitable for the therapy of mammals, preferably of humans suffering from stroke or give reason to suspect a stroke.

As one aspect of the invention, a method is provided for treating stroke of a mammalian subject, comprising the steps (a) starting the administration of G-CSF or a functionally active G-CSF derivative in a therapeutically active amount to the subject, and subsequently (b) administering to the subject a thrombolytic agent in a therapeutically active amount.

As another aspect of the invention, a method is provided for treating stroke of a mammalian subject, comprising the steps (a) administering to a subject G-CSF or a functionally active G-CSF derivative in a therapeutically active amount, and subsequently (b) administering to the subject a thrombolytic agent in a therapeutically active amount.

The mammalian subject can be a human being.

As one embodiment of the invention, a method as mentioned above is provided, wherein the subject undergoes after step (a) and before step (b) a diagnostic examination to exclude the risk of hemorrhagic or other adverse side effects during step (b).

The “thrombolytic agent” of above mentioned step (b) is meant to refer to any agent capable of dissolving at least partially a fibrin-platelet clot. Examples of thrombolytic agents include streptokinase, prourokinase, urokinase, desmoteplase and tissue-type plasminogen activator (t-PA). Although natural t-PA may be employed, it is preferable to employ recombinant t-PA (rt-PA, e.g. Alteplase). The invention may additionally employ hybrids, physiologically active fragments or mutant forms of the above thrombolytic agents. The term “tissue-type plasminogen activator” as used herein is intended to include such hybrids, fragments and mutants, as well as both naturally derived and recombinantly derived tissue-type plasminogen activator.

As a further embodiment of the invention, a method as mentioned above is provided, wherein administration of said G-CSF or functionally active G-CSF derivative of step (a) starts within the first 6 h after onset of the stroke and/or administration of said thrombolytic agent of step (b) starts within the first 24 h after onset of the stroke or in the time period between 4.5 h and 24 h after onset of the stroke or between 6 h and 24 h after stroke.

As a still further embodiment of the invention, a method as mentioned above is provided, wherein G-CSF or functionally active G-CSF derivative of step (a) is administered to the subject within the first 6 h, the first 4.5 h, or the first 3 h after onset of the stroke and/or administration of said thrombolytic agent of step (b) starts within the first 24 h after onset of the stroke or in the time period between 4.5 h and 24 h after onset of the stroke or between 6 h and 24 h after stroke.

As one embodiment of the invention, a method as mentioned above is provided, wherein there is a time period of at least 0.5 h, at least 1.5 h, or at least 3 h between the administration, the start of the administration, or the end of the administration of G-CSF or functionally active G-CSF derivative of step (a) and the start of the administration of said thrombolytic agent of step (b). Preferably, this time period is used for diagnostic examination of the subject, assessing the risk of hemorrhagic or other adverse side effects of the thrombolytic therapy.

As a further aspect of the invention, a method is provided of treating a mammalian subject suffering from acute stroke, comprising an initial G-CSF administration or a start of a initial G-CSF administration, followed by diagnostic examinations, whereas said examinations assess the risk of a thrombolytic therapy for the subject, and, optionally, based on the results of the diagnostic examination, followed by a thrombolytic treatment. Such diagnostic examinations can be e.g. the exclusion of a hemorrhagic stroke, which is a counter-indication for a thrombolytic therapy.

As another embodiment of the invention, a method as mentioned above is provided, wherein said G-CSF of step (a) is given intravenously or subcutaneously in doses of at least 10 μg/kg body weight, at least 90 μg/kg body weight, or at least 130 μg/kg body weight.

As a further aspect of the invention, G-CSF or functionally active derivative thereof is provided for the preparation of a pharmaceutical composition for treating a mammalian subject suffering from acute stroke, wherein the subject is admitted to a stroke unit or a clinic within the initial 6 h after stroke onset or within the time period of 3 to 6 h after stroke onset or within the time period of 4.5 to 6 h after stroke onset, and wherein the expenditure of time for the diagnostic examination necessary to assess the subject's risk of hemorrhagic or other sever adverse side effects of a thrombolytic treatment would otherwise cause the expiration of the therapeutic window for thrombolytic treatment. The thrombolytic treatment in this context can be e.g. the administration of t-PA, such as rt-PA. The therapeutic window for thrombolytic treatment in this context can be within 3 h, within 4.5 h, or within 6 h after stroke onset. The diagnostic examination in this context can last at least 0.5 h, at least 1.5 h, or at least 3 h. The mammalian subject in this context can receive the G-CSF or functionally active derivative thereof immediately after admittance to the stroke unit or clinic, or within the first 6 h, within the first 4.5, or within the first 3 h after stroke onset. Further, the mammalian subject in this context can receive subsequently the thrombolytic treatment if the diagnostic examination permits such a treatment. The mammalian subject in this context can be a human being. The G-CSF in this context can be human G-CSF, preferably, Filgrastim.

The diagnostic examination of above described embodiments is meant to refer to any examination of the mammalian patient suffering from acute stroke which allows, improves, or supports the decision, whether a thrombolytic treatment, particularly thrombolytic treatment with t-PA, of the patient is indicated or contra-indicated. Such diagnostic examinations can be e.g., but without any claim of completeness: Medical imaging such as magnetic resonance imaging (MRI), analysis of blood parameters such as coagulation factors, or also survey of the patients anamnesis. Since patients suffering from acute stroke are frequently unconscious or confused, a survey of the patients anamnesis can be time consuming. Contraindications for thrombolytic treatment of acute stroke, particularly for t-PA treatment, which should be excluded by diagnostic examinations prior starting the treatment are e.g., but without any claim of completeness: Active internal bleeding, history of cerebrovascular accident, recent intracranial or intraspinal surgery or trauma, Intracranial neoplasm, arteriovenous malformation, or aneurysm, bleeding diathesis (including but not limited to current use of oral anticoagulants (e.g., warfarin sodium), an International Normalized Ratio (INR) >1.7, a prothrombin time (PT) >15 seconds, administration of heparin within 48 hours preceding the onset of stroke and elevated activated partial thromboplastin time (aPTT) at presentation, or platelet count <100,000/mm³), uncontrolled hypertension at time of treatment (e.g., >185 mm Hg systolic or >110 mm Hg diastolic), intracranial hemorrhage, subarachnoid hemorrhage, recent (within 3 months) intracranial or intraspinal surgery, serious head trauma, previous stroke, history of intracranial hemorrhage, seizure at the onset of stroke

Granulocyte-colony stimulating factor (G-CSF) is a well known growth factor. The G-CSF that can be employed in the inventive methods described herein are human G-CSF (pro-form, short splice variant (SEQ ID NO: 2), mature form, short splice variant (SEQ ID NO: 4), pro-form, long splice variant (SEQ ID NO: 6), mature form, long splice variant (SEQ ID NO: 8), Filgrastim (SEQ ID NO: 10)) or various functional variants, muteins, and mimetics that are known and available. In the discussion that follows these are referred to as G-CSF derivatives.

Said G-CSF derivatives which can be employed in the present invention are proteins that are at least 70%, preferably at least 80%, more preferably at least 90% identical to human G-CSF amino acid sequences described herein. In another embodiment, the G-CSF that can be used are those that are encoded by polynucleotide sequence with at least 70%, preferably 80%, more preferably at least 90%, 95%, and 97% identity to the human G-CSF coding sequence (pro-form, short splice variant (SEQ ID NO: 1), mature form, short splice variant (SEQ ID NO: 3), pro-form, long splice variant (SEQ ID NO: 5), mature form, long splice variant (SEQ ID NO: 7), Filgrastim (SEQ ID NO: 9)), these polynucleotides will hybridize under stringent conditions to the coding polynucleotide sequence of the human G-CSF coding sequence. The terms “stringent conditions” or “stringent hybridization conditions” includes reference to conditions under which a polynucleotide will hybridize to its target sequence, to a detectably greater degree than other sequences (e.g., at least 2-fold over background). Stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides), for example, high stringency conditions include hybridization in 50% formamide, 1 M NaCl, 1 SDS at 37° C., and a wash in 0.1×SSC at 60 to 65° C. (see Tijssen, Laboratory Techniques in Biochemistry and Molecular Biology-Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995)). Amino acid and polynucleotide identity, homology and/or similarity can be determined using the ClustalW algorithm, MEGALIGN™, Lasergene, Wis.)

Examples of the various G-CSF functional variants, muteins, and mimetics include functional fragments and variants (e.g., structurally and biologically similar to the wild-type protein and having at least one biologically equivalent domain), chemical derivatives of G-CSF (e.g., containing additional chemical moieties, such as polyethyleneglycol and polyethyleneglycol derivatives thereof, and/or glycosylated forms such as Lenogastrim™), and peptidomimetics of G-CSF (e.g., a low molecular weight compound that mimics a peptide in structure and/or function (see, e.g., Abell, Advances in Amino Acid Mimetics and Peptidomimetics, London: JAI Press (1997); Gante, Angew Chem. 1994, 106:1780; Olson et al., J Med Chem. 1993, 36:3039).

Additional examples of G-CSF derivatives include a fusion protein of albumin and G-CSF (Albugranin™), or other fusion modifications such as those disclosed in U.S. Pat No. 6,261,250; PEG-G-CSF conjugates and other PEGylated forms; those described in WO 00/44785 and Viens et al., J Clin Oncology 2002, 6:24; norleucine analogues of G-CSF, those described in U.S. Pat. No. 5,599,690; G-CSF mimetics, such as those described in WO 99/61445, WO 99/61446, and Tian et al., Science 1998, 281:257; G-CSF muteins, where single or multiple amino acids have been modified, deleted or inserted, as described in U.S. Pat. Nos. 5,214,132 and 5,218,092; those G-CSF derivatives described in U.S. Pat. No. 6,261,550 and U.S. Pat. No. 4,810,643; and chimeric molecules, which contain the full sequence or a portion of G-CSF in combination with other sequence fragments, e.g. Leridistim—see, for example, Streeter et al., Exp Hematol. 2001, 29:41, Monahan et al., Exp Hematol. 2001, 29:416, Hood et al., Biochemistry 2001, 40:13598, Farese et al., Stem Cells 2001, 19:514, Farese et al., Stem Cells 2001, 19:522, MacVittie et al., Blood 2000, 95:837. Additionally, the G-CSF derivatives include those with the cysteines at positions 17, 36, 42, 64, and 74 of SEQ ID NO: 4 or analogously of SEQ ID NO: 10, substituted with another amino acid, (such as serine) as described in U.S. Pat. No. 6,004,548, G-CSF with an alanine in the first (N-terminal) position; the modification of at least one amino group in a polypeptide having G-CSF activity as described in EP 0 335 423; G-CSF derivatives having an amino acid substituted or deleted in the N-terminal region of the protein as described in EP 0 272 703; derivatives of naturally occurring G-CSF having at least one of the biological properties of naturally occurring G-CSF and a solution stability of at least 35% at 5 mg/ml in which the derivative has at least Cys¹⁷ of the native sequence replaced by a Ser¹⁷ residue and Asp²⁷ of the native sequence replaced by a Ser²⁷ residue as described in EP 0 459 630; a modified DNA sequence encoding G-CSF where the N-terminus is modified for enhanced expression of protein in recombinant host cells, without changing the amino acid sequence of the protein as described in EP 0 459 630; a G-CSF which is modified by inactivating at least one yeast KEX2 protease processing site for increased yield in recombinant production using yeast as described in EP 0 243 153; lysine altered proteins as described in U.S. Pat. No. 4,904,584; cysteine altered variants of proteins as described in WO 90/12874 (U.S. Pat. No. 5,166,322); the addition of amino acids to either terminus of a G-CSF molecule for the purpose of aiding in the folding of the molecule after prokaryotic expression as described in AU-A-10948/92; substituting the sequence Leu-Gly-His-Ser-Leu-Gly-Ile (SEQ ID NO: 16) at position 50-56 of G-CSF of SEQ ID NO: 4 and position 53 to 59 of the G-CSF of SEQ ID No: 8 or/and at least one of the four histedine residues at positions 43, 79,156 and 170 of the mature G-CSF of SEQ ID NO: xx (174 form) or at positions 46, 82, 159, or 173 of the mature G-CSF of SEQ ID NO: 8 as described in AU-A-763 80/91; and a synthetic G-CSF-encoding nucleic acid sequence incorporating restriction sites to facilitate the cassette mutagenesis of selected regions and flanking restriction sites to facilitate the incorporation of the gene into a desired expression system as described in GB 2 213 821. Further examples of G-CSF analogs include SEQ ID NO: 17) and others described in U.S. Pat. No. 6,632,426. The contents of the above are incorporated herein by reference.

The various functional derivatives, variants, muteins and/or mimetics of G-CSF preferably retain at least 20%, preferably 50%, more preferably at least 75% and/or most preferably at least 90% of the biological activity of wild-type mammalian G-CSF activity—the amount of biological activity include 25%, 30%, 35%, 40%, 45%, 55%, 60%, 65%, 70%, 75%, 80%, 85%, 95%; and all values and subranges there between. Furthermore, the functional derivatives, variants, muteins and/or mimetics of G-CSF can also have 100% or more of the biological activity relative to wild-type mammalian G-CSF activity—the amount of biological activity including at least 105%, at least 110%), at least 125%, at least 150%, and at least 200%.

To measure the biological activity of G-CSF, several known assays can be employed singularly or in combination. One example of determining G-CSF function is illustrated in Example 1. Other methods for determining G-CSF function are known and include a colony formation assay employing murine bone marrow cells; stimulation of proliferation of bone marrow cells induced by G-CSF; specific bioassays with cells lines that depend on G-CSF for growth or that respond to G-CSF (e.g., AML-193; 32D; BaF3; GNFS-60; HL-60; Ml; NFS-60; OCl/AMLIa; and WEHI-3B). These and other assays are described in Braman et al., Am J Hematology 1992, 39:194; Clogston et al., Anal Biochem. 1992, 202:375; Hattori et al., Blood 1990, 75:1228; Kuwabara et al., J Pharmacobiodyn. 1992, 15:121; Motojima et al., J Immunological Methods 1989, 118:187; Sallerfors and Olofsson, Eur J Haematology 1992, 49:199; Shorter et al., Immunology 1992, 75:468; Tanaka and Kaneko, J Pharmacobiodyn. 1992, 15:359; Tie et al., J Immunological Methods 1992, 149:115; Watanabe et al., Anal Biochem. 1991, 195:38.

In one embodiment, the G-CSF is modified or formulated, or is present as a G-CSF mimetic that increases its ability to cross the blood-brain barrier, or shift its distribution coefficient towards brain tissue. An example of such a modification is the addition of PTD or TAT sequences (Cao et al., J Neurosci. 2002, 22:5423; Mi et al., Mol Ther. 2000, 2:339; Morris et al., Nat Biotechnol. 2001, 19:1173; Park et al., J Gen Virol. 2002, 83:1173). These sequences can also be used in mutated forms, and added with additional amino acids at the amino- or carboxy-terminus of proteins. Also, adding bradykinin, or analogous substances to an intravenous application of any G-CSF preparation will support its delivery to the brain, or spinal cord (Emerich et al., Clin Pharmacokinet. 2001, 40:105; Siegal et al., Clin Pharmacokinet. 2002, 41:171).

In one embodiment the biological activity of G-CSF is enhanced by fusion to another hematopoietic factor. The enhanced activity can be measured in a biological activity assay as described above. Such a preferred modification or formulation of G-CSF leads to an increased antiapoptotic effect and/or an increase in neurogenesis. An example for such a modification is Myelopoietin-1, a G-CSF/IL-3 fusion protein (McCubrey et al., Leukemia 2001, 15:1203) or Progenipoietin-1 (ProGP-1) is a fusion protein that binds to the human fetal liver tyrosine kinase flt-3 and the G-CSF receptor.

### EXAMPLES

### Example 1

G-CSF Decreases Infarct Size within Embolic Model

Embolic models of cerebral ischemia possibly present a stroke model that is closer to the human situation compared to the filament model. So far, efficacy of G-CSF has not been shown in embolic models. Here, embolic stroke was modeled by injection of a preformed blood clot into the internal carotid artery of rats.

Male Wistar rats (n=20) weighing approximately 320 g were anesthetized with isoflurane (5% for induction, 2% for surgery, 1.2% for maintenance). PE-50 polyethylene tubing was inserted into the femoral artery for monitoring of mean arterial blood pressure (MABP) and for obtaining blood samples to measure blood gases (pH, PaO₂, PaCO₂), electrolytes (Na⁺, K⁺, Ca²⁺), and plasma glucose. Body temperature was monitored continuously with a rectal probe and maintained at 37.0+/−0.3° C. with a thermostatically controlled heating lamp. For embolic stroke (ES) one red blood clot (diameter=0.35 mm, length=18 mm) was injected into the internal caroted artery (ICA) of 20 animals over approx. 1 s at the bifurcation of the pterygopalatanine artery (PPA) and ICA. Laser Doppler Flowmetry was used to monitor occlusion success.

Verum (G-CSF, Filgrastim (SEQ ID NO: 10)) and vehicle (buffer solution (250 mM Sorbitol, 0.004% Tween-80, and 10 mM sodium-acetate buffer (pH 4)) groups received two injections: an intravenous infusion (120 μg/kg body weight over 30 min) at 1 h after clot injection, and an intraperitoneal bolus (120 μg/kg body weight) at 4h after clot injection. At 24 h animals were neurologically scored as previously described (rating scale: 0: no deficit, 1: failure to extend the left forepaw, 2: decreased grip strength of left forepaw, 3: circling to paretic side by pulling the tail, 4: spontaneous contralateral circling, and 5:death; Menzies et al., Neurosurgery 1992, 31:100) and sacrificed to determine infarct volumes by 2,3,5-triphenyltetrazolium chloride (TTC) staining with edema correction (Meng et al., Ann Neurol. 2004, 55:207).

Physiological parameters (blood pH, partial pressure of blood gases (PaCO₂, PaO₂), plasma concentrations of electrolytes (Na⁺, K⁺, CA²⁺) and of glucose) were not significantly changed by treatment. Also, MABP was not influenced by treatment (p>0.05 by repeated measures ANOVA), however there was a significant group-independent drop in MABP at 30 min, after which the blood pressure rose again.

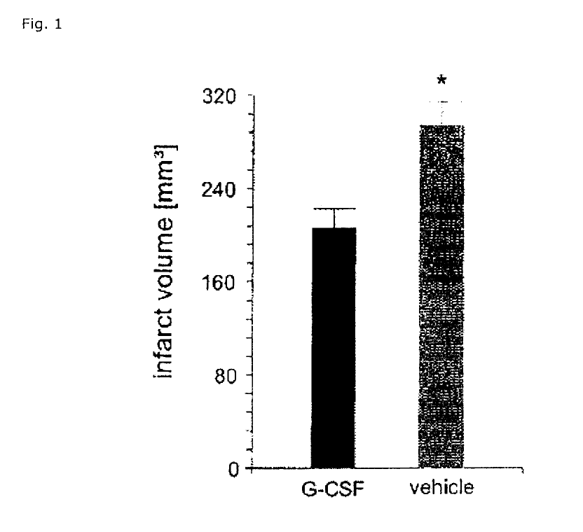

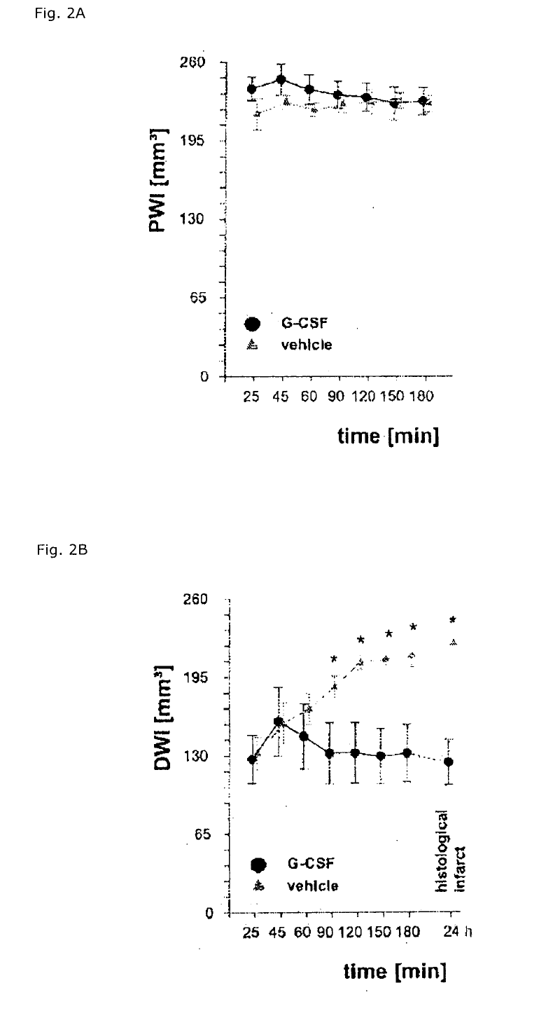

12 of 20 animals died prematurely between 16 and 24 h post ES and were therefore included in the TTC analyses. Infarct volumes determined by postmortem TTC staining were 295+/−20 mm³ (vehicle) vs. 206+/−16 mm³ (G-CSF, means+/−SEM; P=0.003) (FIG. 1). This considerable decrease in infarct size was however not reflected in the neuroscore at 24 h, which did not show any difference between treatments (vehicle: 4.0+/−1.33; G-CSF: 4.2+/−1.32), likely reflecting the insensitivity of that scale for larger infarcts.

### Example 2

G-CSF Halts the Evolution of a DWI Lesion in the Presence of a Permanent Perfusion Deficit

Permanent filament occlusion of the MCA was performed as previously described using 4-0 silicone-coated nylon filament sutures (suture occlusion of the right middle cerebral (sMCAO; Bouley et al., Neurosci Lett. 2007, 412:185). Wistar rats (n=15) weighing 320+/−19 g were anesthetized with isoflurane (5% for induction, 2% for surgery, 1.2% for maintenance) in room air. PE-50 polyethylene tubing was inserted into the femoral artery for monitoring of mean arterial blood pressure (MABP) and for obtaining blood samples to measure blood gases (pH, PaO₂, PaCO₂), electrolytes (Na⁺, K⁺, Ca²⁺), and plasma glucose at prior to as well as 30, 60, 90, 120, 180 min after middle cerebral artery occlusion (MCAO). Body temperature was monitored continuously with a rectal probe and maintained at 37.0+/−0.3° C. with a thermostatically controlled heating lamp.

The perfusion deficit and DWI lesion was monitored over a time period of 180 min by MRI measurements. These MRI experiments were performed on a 4.7 T/40 cm horizontal magnet equipped with a Biospec Bruker console (Billerica, Mass., USA), and a 20 G/cm gradient insert (ID=12 cm, 120 ps rise time). A surface coil (ID=2.3 cm) was used for brain imaging and an actively decoupled neck coil for perfusion labelling (Meng et al., Ann Neurol. 2004, 55:207). Animals were imaged at 25, 45, 60, 90, 120, 150 and 180 min post-sMCAO. Three ADC maps were separately acquired with diffusion-sensitive gradients applied along the x, y, or z direction. Single shot, echo-planar images (EPI) were acquired over 3 min with matrix=64×64, spectral width=200 kHz, TR=2 s (90° flipangle), TE=37.5 ms, b=8 and 1,300 s/mm², Δ=24 ms, δ=4.75 ms, field of view (FOV)=2.56×2.56 cm, seven 1.5 mm slices, and 16 averages. Quantitative CBF measurements were made using the continuous arterial spin-labeling technique with single-shot, gradient-echo, EPI acquisition. Sixty paired images (for signal averaging) were acquired over 4 min, alternately, one with arterial spin labeling and the other (control) without spin-labeling preparation. The MRI parameters were similar to ADC measurements except TE=13.5 milliseconds. Arterial spin labeling utilized a 1.78-second, square radiofrequency pulse in the presence of 1.0 Gauss/cm gradient along the flow direction. The sign of the frequency offset was switched for nonlabeled images.

Final infarct volumes were determined at 24 h after onset of occlusion, whereas brains were removed and sectioned coronally into seven 1.5 mm-thick slices corresponding to the MR slices and stained with TTC.

Rats were treated with vehicle (buffer solution (250 mM Sorbitol, 0.004% Tween-80, and 10 mM sodium-acetate buffer (pH 4)), n=5) or G-CSF (Filgrastim, SEQ ID NO: 10; n=10) at 1 h after occlusion (intravenously; 120 μg/kg body weight over 30 min) and 4 h after occlusion (intraperitoneally; 120 μg/kg body weight as bolus).

Animals surviving for more than 16 hours were prespecified to be included in the study while those dying before 16 hours were excluded. Effects of G-CSF on apparent diffusion coefficient (ADC) and cerebral blood flow (CBF) characteristics as well as the spatiotemporal evolution of the ischemic lesion were evaluated.

Blood gases, electrolytes, pH, and blood glucose levels did not differ between the two groups. MABP was also not significantly different between treatment groups in both experiments (p>>0.05 by repeated measures ANOVA), however there was a group-independent significant rise over the course of the experiment (p<0.05 for factor time by repeated measures ANOVA). 2 of 15 animals died between 16 to 24 h.

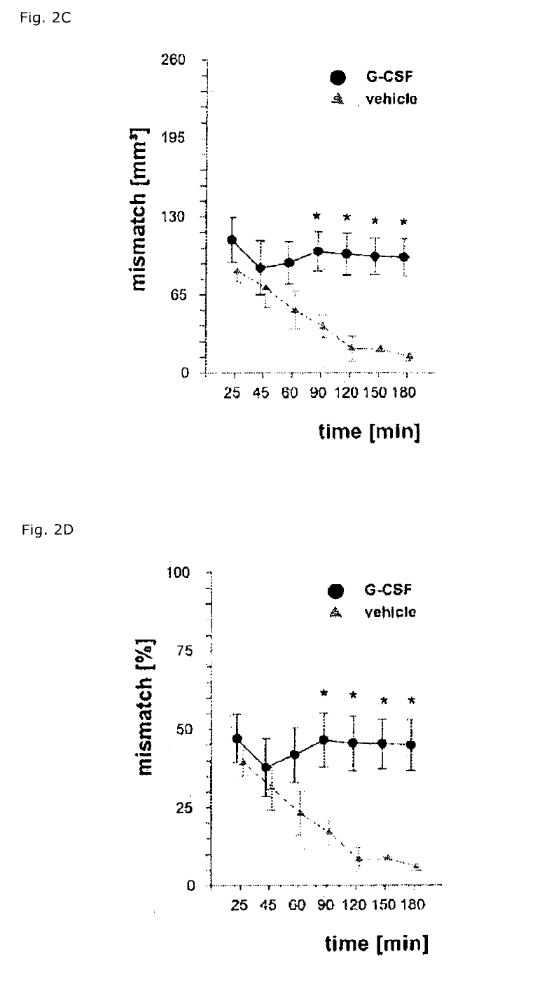

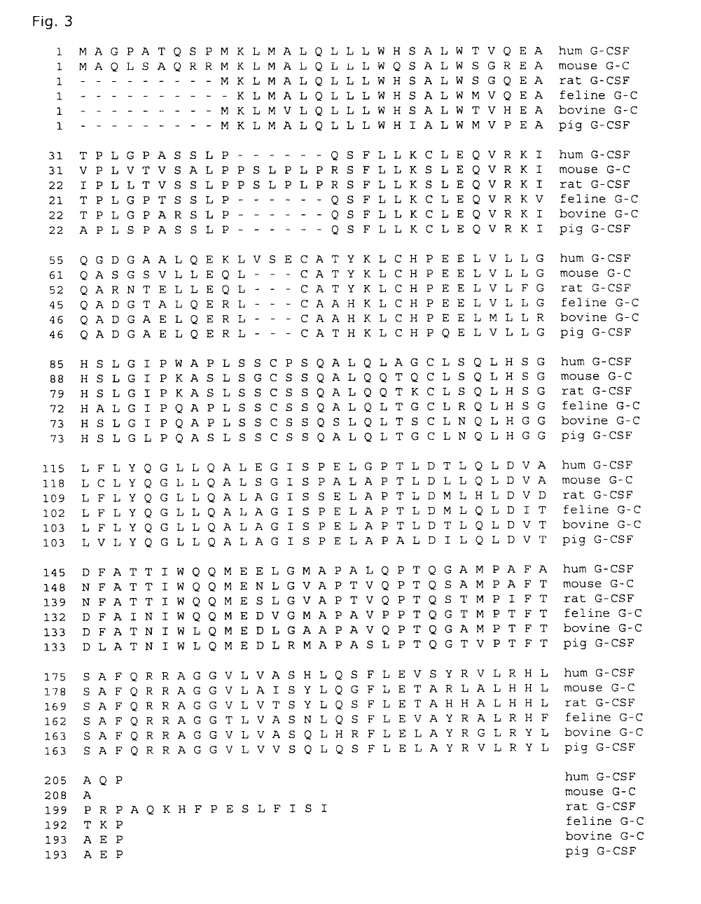

Images were analyzed using Quickvol II (Schmidt et al., J Neurooncol. 2004, 68:207). Quantitative CBF and ADC maps and their corresponding threshold-derived lesion volumes were calculated as described previously (Meng et al., Ann Neurol. 2004, 55:207). The thresholds used to define abnormal DWI and PWI regions were a reduction to 0.53×10⁻³ mm²/s for ADC and 0.3 mL/g/min for CBF as previously validated (Meng et al., Ann Neurol. 2004, 55:207). FIG. 2 summarizes the spatiotemporal evolution of threshold-derived ADC and CBF lesion volumes. The CBF lesion volume did not differ between groups (vehicle and G-CSF) and remained relatively constant over time at about 230 mm³ (FIG. 2A).

The ADC-derived lesion in the vehicle-treated animals increased with time in a linear fashion until 120 min, when the curve flattened. The final infarct volume determined at 24 h by the TTC method lay slightly above the last DWI volume measured at 180 min post occlusion. In G-CSF-treated animals, the DWI lesion grew from 25 min to 45 min post occlusion identical to the vehicle situation. However, when the MRI data were obtained at the 60 min time point after application of G-CSF, the increase seemed to begin to reverse. At 90 min, the DWI lesion in the G-CSF-treated animals became significantly smaller compared to the vehicle-treated rats (repeated measures ANOVA: p<0.0001 for the interaction treatment-time followed by Tukey-Kramer post-hoc test). For the following time points measured, the lesion remained stable until the end of the MRI data acquisition at 180 min, and resulted in a final infarct at 24 h of approximately the same size (FIG. 2B).

The TTC-defined infarct volumes were significantly different between the treatment groups (223+/−7 mm³ (vehicle) vs. 124+/−19 mm³ (G-CSF; p=0.007), and correspond well to the 3 h ADC lesion volumes in both groups and to the 3 h CBF in the vehicle group (FIGS. 2B and 2A).

FIGS. 2C and 2D show the absolute and relative mismatch between CBF and ADC derived volumes. All two measures also became significantly different at 90 min following occlusion (p<0.05; repeated measures ANOVA followed by Tukey Kramer post hoc test). Employing an alternative statistical approach and comparing DWI volume behaviour over time relative to PWI volume and treatment by a multiple linear regression model (factors: PWI, ANIMAL (random factor), TREATMENT, TIME, TIME×TREATMENT interaction) showed the treatment effect to become significant at 84 min post sMCAO. The present experiment shows that the action of G-CSF must be immediate to allow for a significant effect on the DWI deficit volume at least at 90 min post onset of occlusion. Induction of anti-apoptotic cascades in vitro is immediate, with phosphorylation and activation of Akt within 5 min after addition of G-CSF to the neurons 9. In contrast, an indirect effect mediated by bone-marrow derived cells would require release of those cells from the bone marrow into the bloodstream, passage of the blood-brain barrier, and tissue invasion, possibly followed by release of protective factors. This is unlikely to be rapid enough for the effect observed in the current experiment.

Significant between-group differences were not detected in Menzies neurological scores at 4 and 24 h, respectively, likely reflecting the insensitivity of that scale for larger infarcts.

1. A method for treating stroke of a mammalian subject, comprising:
(a) starting the administration of G-CSF or a functionally active G-CSF derivative in a therapeutically active amount to the subject; and subsequently
(b) administering to the subject a thrombolytic agent in a therapeutically active amount.

2. The method of claim 1, wherein the subject undergoes after the starting (a) and before the administering (b) a diagnostic examination to exclude the risk of hemorrhagic or other adverse side effects during the administering (b).

3. The method of claim 1, wherein human G-CSF is used administered in (a).

4. The method of claim 1, wherein the thrombolytic agent in (b) comprises rt-PA.

5. The method of claim 1, wherein the administration of G-CSF or a functionally active G-CSF derivative starts within the first 6 h after onset of the stroke.

6. The method of claim 1, wherein the thrombolytic agent is administered later than 6 h after onset of the stroke.

7. The method of claim 1, wherein the administration of G-CSF or a functionally active G-CSF derivative is completed within the first 6 h after onset of the stroke.

8. The method of claim 1, wherein the thrombolytic agent is administered at least 0.5 h after the starting of the administration of G-CSF or a functionally active G-CSF derivative.

9. The method of claim 2, wherein the diagnostic examination lasts at lest least 0.5 h.

10. The method of claim 1, wherein G-CSF is given intravenously or subcutaneously in a dose of at least 90 μg/kg body weight.

11. The method of claim 1, wherein the stroke is an acute stroke,
wherein the subject is admitted to a stroke unit or a clinic within the initial 6 h after stroke onset, and
wherein an expenditure of time for a diagnostic examination necessary to assess the subject's risk of hemorrhagic or at least one other severe adverse side effect of a thrombolytic treatment would otherwise cause an expiration of a therapeutic window for thrombolytic treatment.

12. The method of claim 11, wherein the therapeutic window for thrombolytic treatment is 3 h after stroke onset.

13. The method of claim 11, wherein the diagnostic examination lasts at least 0.5 h

14. The method of claim 11, wherein the mammalian subject receives the G-CSF or functionally active G-CSF derivative immediately after admittance to the stroke unit or clinic.

15. The method of claim 11, wherein the G-CSF is human G-CSF.

16. The method of claim 1, wherein a stroke onset has been observed in the subject within six hours before the administration of G-CSF and wherein apart from observing the stroke onsetₛ no further stroke diagnosis has been performed before the administration of G-CSF.

17. The method of claim 1, wherein a stroke onset has been observed in the subject within six hours before the administration of G-CSF and wherein after the administration of G-CSF a diagnostic examination of the subject to exclude the risk of hemorrhagic or other adverse side effects due to the administration of the thrombolytic agent is performed,

18. A thrombolytic agent, suitable for treating stroke in a subject, wherein the subject has been administered with G-CSF within 6 hours after the onset of stroke in the subject and wherein after administration of the G-CSF a diagnostic examination of the subject to exclude a risk of hemorrhagic or at least one other adverse side effect due to the administration of the thrombolytic agent has been performed.

19. The thrombolytic agent of claim 18, suitable for administration later than 6 h after onset of the stroke.

20. A composition, comprising:
a G-CSF or a functionally active G-CSF derivative; and
a pharmaceutically acceptable carrier.
